(19) United States
(12) Patent Application Publication
(10) Pub. No.: US 2009/0069334 A1
(43) Pub. Date: Mar. 12, 2009

(54) Pyridazine based alpha-helix mimetics

(75) Inventors: Julius Rebek, JR., La Jolla, CA (US); Shannon Biros, Grand Haven, MI (US); Lionel Moisan, Thonon-les-Bains (FR); Alessandro Volonterio, Milano (IT); Enrique Mann, Madrid (ES); Trevor Dale, San Diego, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA (US)

(21) Appl. No.: 12/228,559

(22) Filed: Aug. 13, 2008

Related U.S. Application Data
(60) Provisional application No. 60/965,100, filed on Aug. 18, 2007.

(51) Int. Cl. A61K 31/501; C07D 403/14; A61P 43/00

(52) U.S. Cl. 514/252.02; 544/238

(57) ABSTRACT

The synthesis of new α-helix scaffolds mimicking i, i+3 or i+4, i+7 residues, was accomplished. The common pyridazine heterocycle originates from the easily available building block, 6. These scaffolds may be thought of as synthetic counterparts of amphiphilic α-helices having a “wet face” along one side and a hydrophobic face along the other side of the helix.

### FIELD OF INVENTION

The present invention is directed to nonpeptidic scaffolds that serve as alpha-helix mimetics. More particularly, the invention is directed to compounds, intermediates and methods for the preparation and uses thereof, and pharmaceutical compositions comprising nonpeptidic scaffolds having a pyridazine core. The novel compounds are useful as alpha-helix mimetics for efficiently disrupting protein-protein interactions such as Bak/Bcl-X_{L}, p53/HDM2, calmodulin/smooth muscle myosin light-chain kinase, and gp41 assembly. Methods for treating diseases or conditions which are modulated through disruption of interactions between alpha helical proteins and their binding sites are other aspects of the invention

### BACKGROUND

α-Helices are the most common protein secondary structures and play a pivotal role in many protein-protein interactions. It is a rod-like structure wherein the polypeptide chain coils around like a corkscrew to form the inner part of the rod and the side chains extend outward in a helical array. Approximately 3.6 amino acid residues make up a single turn of an alpha-helix; thus the side chains that are adjacent in space and make up a “side” of an alpha-helix occur every three to four residues along the linear amino acid sequence. The alpha-helix conformation is stabilized by steric interactions along the backbone as well as hydrogen bonding interactions between the backbone amide carbonyls and NH groups of each amino acid. Frequently the critical interactions are found along a “face” of the helix involving side chains from the i, i+3 or i+4 and i+7 residues. These project from the α-helix with well known distances and angular relationships (Fairlie, D. P.; et al. Curr. Med. Chem. 1998, 5, 29-62; Jain, R.; et al. Mol. Divers. 2004, 8, 89-100; Cochran, A. G. Curr. Opin. Chem. Biol. 2001, 5, 654-659; Zutshi, R.; et al. Curr. Opin. Chem. Biol. 1998, 2, 62-66; Toogood, P. L. J. Med. Chem. 2002, 5, 1543-1558; Berg, T. Angew. Chem. Int Ed. 2003, 42, 2462-2481.).

Molecules that can predictably and selectively reproduce these projections could be valuable as tools in molecular biology and, potentially, as leads in drug discovery (Walensky, L. D.; et al. Science 2004, 305, 1466-1470). Nearly a third of the residues in known proteins form alpha-helices and such helices are important structural elements in various biological recognition events, including ligand-receptor interactions, protein-DNA interactions, protein-RNA interactions, and protein-membrane interactions. Given the importance of alpha-helices in biological systems, it would be desirable to have available small organic molecules that act as mimics of alpha-helices. Such compounds would be useful not only as research tools, but as therapeutics to treat conditions mediated by alpha-helix binding enzymes and receptors.

Side chains in positions i, i+3li+4, i+7, and i+11 appear on the same face of the helix are frequently crucial for the interaction (Davis, J. M.; et al. Chem. Soc. Rev. 2007, 36, 326; Fletcher, S.; Hamilton, A. D. J. R. Soc. Interface 2006, 3, 215; Yin, H.; Hamilton, A. D. Angew. Chem. Int. Ed. 2005, 44, 4130; Jain, R.; et al. Mol. Divers. 2004, 8, 89; Peczuh, M. W.; Hamilton, A. D. Chem. Rev. 2000, 100, 2479.). Hamilton and co-workers pioneered the synthesis of non-peptidic α-helix mimetics based on terphenyl, terephthalamide, and oligopyridine scaffolds that display side chains in a manner that closely resembles those in position i, i+4, and i+7 of an α-helix (Kutzki, O.; et al. J. Am. Chem. Soc. 2002, 124, 11838; Ernst, J. T.; et al. Angew. Chem. Int. Ed. 2003, 42, 535; Yin, H.; et al. J. Am. Chem. Soc. 2005, 127, 5463.). They were shown to efficiently disrupt protein-protein interactions such as Bak/Bcl-X_{L} (Yin, H.; et al. J. Am. Chem. Soc. 2005, 127, 10191.), p53/HDM2 (Yin, H.; et al. Angew. Chem. Int. Ed. 2005, 44, 2704.), calmodulin/smooth muscle myosin light-chain kinase (Orner, B. P.; et al. J. Am. Chem. Soc. 2001, 123, 5382.), and gp41 assembly (Ernst, J. T.; et al. Angew. Chem. Int. Ed. 2002, 41, 278.). During efforts towards the design of inhibitors of protein-protein interactions (Davis, C. N.; et al. Proc. Natl. Acad. Sci. USA 2006, 103, 2953; Bartfai, T.; et al. Proc. Natl. Acad. Sci. USA 2004, 101, 10470; Bartfai, T; et al. Proc. Natl. Acad. Sci. USA 2003, 100, 7971.), methodology was developed for structurally similar molecules featuring more hydrophilic components and a facile synthetic route (Biros, S. M.; et al. Bioorg. Med. Chem. Lett. 2007, 17, 4641.).

The syntheses of peptidomimetics having a stabilized α-helical conformation have been achieved by introducing synthetic templates into the peptidic chain (Kemp, D. S.; et al. J. Am. Chem. Soc. 1996, 118, 4240-4248; Austin, R. E.; et al. J. Am. Chem. Soc. 1997, 119, 6461-6472), by using β-hairpin mimetics (Fasan, R.; et al. Angew. Chem. Int. Ed. 2004, 43, 2109-2112), β-peptide sequences (Kritzer, J. A.; et al. J. Am. Chem. Soc. 2004, 126, 9468-9469), and unnatural oligomers with discrete folding propensities (foldamers) (Sadowsky, J. D.; et al. J. Am. Chem. Soc. 2005, 127, 11966-11968). Small synthetic molecules able to mimic the surfaces of constrained peptides offer the advantage of improved stability, lower molecular weight and in some cases better bioavailability. Although synthetic small molecules that adopt various well-defined secondary structures are well-documented (Hagihara, M.; et al. J. Am. Chem. Soc. 1992, 114, 6568-6570; Gennari, G.; et al. Angew. Chem. Int. Ed. Engl. 1994, 33, 2067-2069; Gude, M.; et al. Tetrahedron Lett. 1996, 37, 8589-8592; Cho, C. Y.; et al. Science 1993, 261, 1303-1305; Hamuro, Y.; et al. J. Am. Chem. Soc. 1996, 118, 7529-7541; Nowick, J. S.; et al. J. Am. Chem. Soc. 1996, 118, 1066-1072; Lokey, R. S.; Iverson, B. L. Nature, 1995, 375, 303-305; Murray, T. J.; Zimmerman S. C. J. Am. Chem. Soc. 1992, 114, 4010-4011; Antuch, W.; et al. Bioorg. Med. Chem. Lett. 2006, 16, 1740-1743. For reviews concerning α-helix mimetics, see: Yin, H.; Hamilton, A. D. Angew. Chem. Int. Ed. 2005, 44, 4130-4163; Fletcher, S.; Hamilton, A. D. J. R. Soc. Interface 2006, 3, 215-233; Davis, J. M.; et al. Chem. Soc. Rev. 2007, 36, 326-334. See also: Cummins, M. D.; et al. Chem. Biol. Drug Des. 2006, 67, 201-205; Ahn, J-M. Han, S-Y. Tetrahedron Lett. 2007, 48, 3543-3547), the first useful mimetics for an α-helix were reported only recently by Hamilton and coworkers: the terphenyl scaffold (Orner, B. P.; et al. J. Am. Chem. Soc. 2001, 123, 5382-5383; Yin, H.; et al. J. Am. Chem. Soc. 2005, 127, 10191-10196; Yin, H.; et al. Angew. Chem. Int. Ed. 2005, 44, 2704-2707), and its pyridine (Ernst, J. T.; et al. Angew. Chem. Int. Ed. 2003, 42, 535-539) and terephthalic acid (Yin, H.; Hamilton, A. D. Bioorg. Med. Chem. Lett. 2004, 14, 1375-1379) analogues.

Bak and Bcl-x_{L} belong to the Bcl-2 family of proteins, which regulate cell death through an intricate balance of homodimer and heterodimer complexes formed within this class of proteins (M. C. Raff, Science 1994, 264, 668-669; D. T. Chao, S. J. Korsmeyer, Annu. Rev. Immunol. 1998, 16, 395-419; C. B. Thompson, Science 1995, 267, 1456-1462; L. L. Rubin, K. L. Philpott, S. F. Brooks, Curr. Biol. 1993, 3, 391-394). Overexpression of anti-apoptotic proteins such as Bcl-x_{L} and Bcl-2 prevent cells from triggering programmed death pathways and has been linked to a variety of cancers. Bcl-2 protein plays a critical role in inhibiting anticancer drug-induced apoptosis, which is mediated by a mitochondria-dependent pathway that controls the release of cytochrome c from mitochondria through anion channels. Constitutive overexpression of Bcl-2 or unchanged expression after treatment with anticancer drugs confers drug resistance not only to hematologic malignancies but also to solid tumors (R. Kim et al. Cancer 2004, 101, 2491-2502). A current strategy for developing new anticancer agents is to identify molecules that bind to the Bak-recognition site on Bcl-x_{L}, disrupting the complexation of the two proteins and therefore antagonizing Bcl-x_{L} function (O. Kutzki et al. J. Am. Chem. Soc. 2002, 124, 11, 832-11, 839). The structure determined by NMR spectroscopy (M. Sattler et al. Science 1997, 275, 983-986) shows the 16 residue BH3 domain peptide from Bak (aa 72 to 87, K_{d}≈300 nM) bound in a helical conformation to a hydrophobic cleft on the surface of Bcl-x_{L}, formed by the BH1, BH2, and BH3 domains of the protein. The crucial residues for binding were shown by alanine scanning to be V74, L78, I81, and I85, which project in an i, i+4, i+7, i+11 arrangement from one face of the α-helix. The Bak peptide is a random coil in solution but adopts an α-helical conformation when complexed to Bcl-x_{L}. Studies utilizing stabilized helices of the Bak BH3 domain have shown the importance of this conformation for tight binding. (J. W. Chin, A. Schepartz, Angew. Chem. 2001, 113, 3922-3925; Angew. Chem. Int. Ed. 2001, 40, 3806-3809.)

Small molecule mimetics of alpha-helices are of immense pharmaceutical interest and would circumvent the problems associated with the use of peptidic agents. Accordingly, there is a need in the art for small molecule compounds that can modulate the activity of alpha-helix mediated interactions and therefore would be useful in the treatment of a variety of diseases mediated by these proteins.

Disclosed herein is a new class of low-molecular-weight α-helix mimetics featuring a pyridazine ring and hydrophobic amino-acid side chains.

### SUMMARY

A first aspect of the invention is directed to a nonpeptidic mimetic of the i, i+3 or i+4, and i+7 positions of a peptide alpha-helix. This nonpeptidic mimetic is represented by Formula (I):

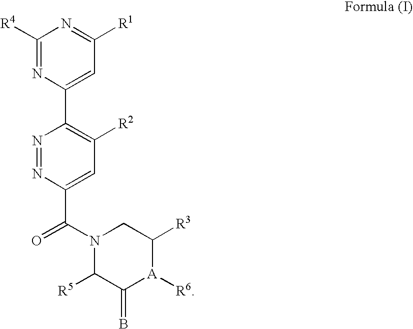

In Formula (I), at least one of R¹ and R⁴ is a side chain of a naturally occurring amino acid or homolog thereof corresponding to the i position of the peptide alpha helix. R² is a side chain of a naturally occurring amino acid or homolog thereof corresponding to the i+3 or i+4 positions of the peptide alpha helix, or, alternatively is a radical selected from the group of radicals consisting of —(C₁-C₉ alkyl), —CH₂(C₃-C₈ cycloalkyl), and —CH₂(C₆-C₁₀ aryl). At least one of R³ and R⁵ is a side chain of a naturally occurring amino acid or homolog thereof corresponding to the i+7 position of the peptide alpha helix. If R¹ is not a side chain of a naturally occurring amino acid or homolog thereof corresponding to the i position of the peptide alpha helix, then R¹ is a radical selected from the group of radicals consisting of —H, —OH, —(C₁-C₉ alkyl), —CH₂(C₃-C₈ cycloalkyl), and —CH₂(C₆-C₁₀ aryl). If R⁴ is not a side chain of a naturally occurring amino acid or homolog thereof corresponding to the i position of the peptide alpha helix, then R⁴ is selected from the group of radicals consisting of —H, —OH, —O(C₁-C₆ alkyl), —S—(C₁-C₆ alkyl), —NH—(C₁-C₆ alkyl), and —(C₁-C₆ alkyl). If R³ is not a side chain of a naturally occurring amino acid or homolog thereof corresponding to the i+7 position, then R³ is a radical selected from the group consisting of hydrogen, —O(C₁-C₆ alkyl), and —OC(O)—(C₁-C₆ alkyl). R⁶ is either absent or is selected from the group of radicals consisting of —H, —(C₁-C₆ alkyl), —(C₃-C₈ cycloalkyl), —(C₁-C₆ alkylene) COOH, —(C₃-C₈ cycloalkylene) COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH. “A” is selected from the group of di- or triradicals consisting of [═N(CH₃)—]⁺, ═N—, —O—, —CH₂—, and ═CH—. “B” is either —O— or (—H)₂. However, the following provisos apply. At least one of R¹ and R⁴ is a side chain of a naturally occurring amino acid or homolog thereof corresponding to the i position of the peptide alpha helix. At least one of R³ and R⁵ is a side chain of a naturally occurring amino acid or homolog thereof corresponding to the i+7 position of the peptide alpha helix. If R⁶ is absent, then “A” is selected from the group of diradicals consisting of —O—, and —CH₂—. At most, only one of the side chains of the naturally occurring amino acids or homologs thereof corresponding to the i, i+3 or i+4, and i+7 positions of the peptide alpha-helix can be hydrogen. In a preferred embodiment of this first aspect of the invention, the side chain of the naturally occurring amino acid with respect to R¹, R², R³, R⁴, and R⁵ is a radical independently selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof. In another preferred embodiment, the nonpeptidic mimetic is represented by the following structure:

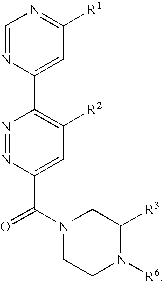

In the above structure, R¹ and R² are radicals independently selected from the group of radicals consisting of —(C₁-C₉ alkyl), —CH₂(C₃-C₈ cycloalkyl), and —CH₂(C₆-C₁₀ aryl); R³ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl); and R⁶ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH. Preferred species of this first aspect of the invention are represented by the following structures:

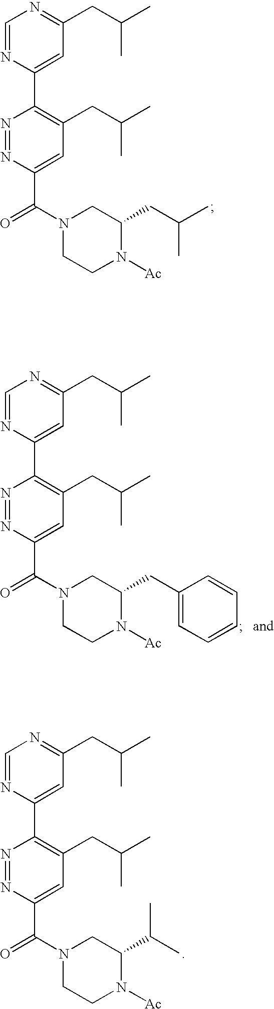

A second aspect of the invention is directed to another nonpeptidic mimetic of the i, i+3 or i+4, and i+7 positions of a peptide alpha-helix. The nonpeptidic mimetic is represented by Formula (II):

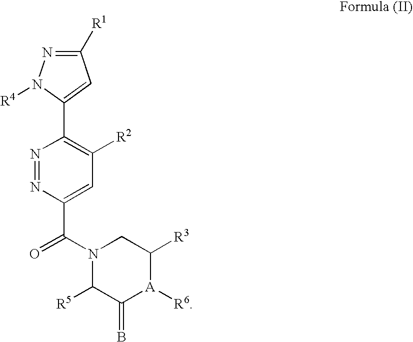

In Formula (II), at least one of R¹ and R⁴ is a side chain of a naturally occurring amino acid or homolog thereof corresponding to the i position of the peptide alpha helix. R² is a side chain of a naturally occurring amino acid or homolog thereof corresponding to the i+3 or i+4 position of the peptide alpha helix, or alternatively, is a radical selected from the group consisting of —(C₁-C₉ alkyl), —CH₂(C₃-C₈ cycloalkyl), and —CH₂(C₆-C₁₀ aryl). At least one of R³ and R⁵ is a side chain of a naturally occurring amino acid or homolog thereof corresponding to the i+7 position of the peptide alpha helix. If R¹ is not a side chain of a naturally occurring amino acid or homolog thereof corresponding to the i position of the peptide alpha helix, then R¹ is a radical selected from the group of radicals consisting of —H, —OH, —(C₁-C₉ alkyl), —CH₂(C₃-C₈ cycloalkyl), and —CH₂(C₆-C₁₀ aryl). If R⁴ is not a side chain of a naturally occurring amino acid or homolog thereof corresponding to the i position of the peptide alpha helix, then R⁴ is selected from the group of radicals consisting of —H, —(C₁-C₆ alkyl), —(C₁-C₆ alkylene)COOH, —C(O)(C₁-C₆ alkyl), —C(O)(C₁-C₆ alkylene)COOH. If R³ is not a side chain of a naturally occurring amino acid or homolog thereof corresponding to the i+7 position of the peptide alpha helix, then R³ is a radical selected from the group consisting of —O(C₁-C₆ alkyl) and —OC(O)—(C₁-C₆ alkyl). R⁵ is either absent or is selected from the group of radicals consisting of —H, —(C₁-C₆ alkyl), —(C₃-C₈ cycloalkyl), —(C₁-C₆ alkylene)COOH, —(C₃-C₈ cycloalkylene) COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH. “A” is selected from the group of di- or triradicals consisting of [═N(CH₃)—]⁺, ═N—, —O—, —CH₂—, and ═CH—. “B” is either —O— or (—H)₂. However, the following provisos apply. At least one of R¹ and R⁴ is a side chain of a naturally occurring amino acid or homolog thereof corresponding to the i position of the peptide alpha helix. At least one of R³ and R⁵ is a side chain of a naturally occurring amino acid or homolog thereof corresponding to the i+7 position of the peptide alpha helix. If R⁶ is absent, then A is selected from the group of diradicals consisting of —O—, and —CH₂—. At most, only one of the side chains of the naturally occurring amino acids or homolog thereof corresponding to the i, i+3 or i+4, and i+7 positions of the peptide alpha-helix can be hydrogen. In a preferred embodiment of this second aspect of the invention, the side chain of the naturally occurring amino acid with respect to R¹, R², R³, R⁴, and R⁵ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof. Another embodiment of this second aspect of the invention is represented by the following structure:

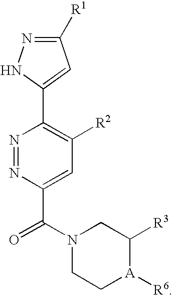

In the above structure, R¹ and R² are radicals independently selected from the group of radicals consisting of —(C₁-C₉ alkyl), —CH₂(C₃-C₈ cycloalkyl), and —CH₂(C₆-C₁₀ aryl). R³ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof. Preferred species of this second aspect of the invention are represented by the following structures:

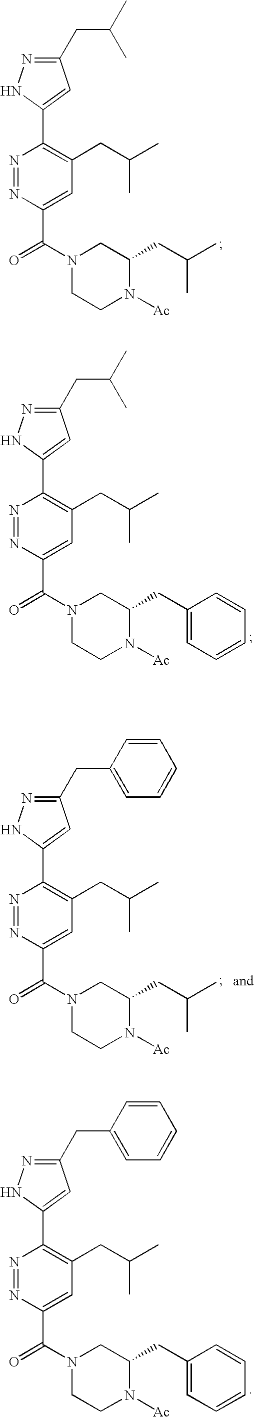

A third aspect of the invention is directed to another nonpeptidic mimetic of the i, i+3 or i+4, and i+7 positions of a peptide alpha-helix. The nonpeptidic mimetic is represented by Formula (III):

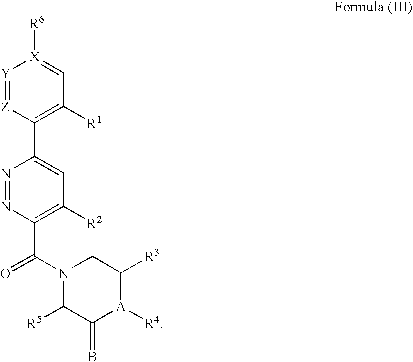

In Formula (III), at least one of R³ and R⁵ is a side chain of a naturally occurring amino acid or homolog thereof corresponding to the i position of the peptide alpha helix. R² is a side chain of a naturally occurring amino acid or homolog thereof corresponding to the i+3 or i+4 position of the peptide alpha helix, or, alternatively, R² is selected from the group of radicals consisting of —(C₁-C₉ alkyl), —CH₂(C₃-C₈ cycloalkyl), and —CH₂(C₆-C₁₀ aryl). R¹ is a side chain of a naturally occurring amino acid or homolog thereof corresponding to the i+7 position of the peptide alpha helix, or, alternatively, is selected from the group of radicals consisting of —(C₁-C₉ alkyl), —CH₂(C₃-C₈ cycloalkyl), —O(C₁-C₉ alkyl), and —OCH₂(C₃-C₈ cycloalkyl). If R³ is not a side chain of a naturally occurring amino acid or homolog thereof corresponding to the i position of the peptide alpha helix, then R³ is a radical is selected from the group consisting of —O(C₁-C₆ alkyl) and —OC(O)—(C₁-C₆ alkyl). R⁴ is selected from the group of radicals consisting of —H, —(C₁-C₆ alkyl), —(C₃-C₈ cycloalkyl), —(C₁-C₆ alkylene)COOH, —(C₃-C₈ cycloalkylene) COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH. R⁶ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH. X, Y, and Z are each independently selected from the group consisting of C and N. A is selected from the group of di- or triradicals consisting of [═N(CH₃)—]⁺, ═N—, —O—, —CH₂—, and ═CH—. B is either —O— or (—H)₂. However, the following provisos apply. At least one of R³ and R⁵ is a side chain of a naturally occurring amino acid or homolog thereof corresponding to the i position of the peptide alpha helix. If R⁶ is absent, then A is selected from the group of diradicals consisting of —O—, and —CH₂—. At most, only one of the side chains of the naturally occurring amino acids or homologs thereof corresponding to the i, i+3 or i+4, and i+7 positions of the peptide alpha-helix can be hydrogen. In a preferred embodiment of this third aspect of the invention, the side chain of the naturally occurring amino acid from which R¹, R², R³, and R⁵ may be selected is a radical independently selected from the group consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl). In another preferred embodiment, the nonpeptidic mimetic is represented by the following structure:

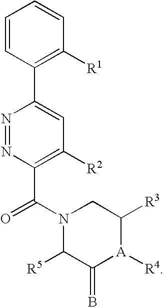

In the above structure, R¹ is independently selected from the group of radicals consisting of —(C₁-C₉ alkyl), —CH₂(C₃-C₈ cycloalkyl), —O(C₁-C₉ alkyl), and —OCH₂(C₃-C₈ cycloalkyl). R² is independently selected from the group of radicals consisting of —(C₁-C₉ alkyl), —CH₂(C₃-C₈ cycloalkyl), and —CH₂(C₆-C₁₀ aryl). R³ is selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof. R⁴ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH. R⁵ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, and homologs thereof. In another preferred embodiment of this third aspect of the invention, the nonpeptidic mimetic is represented by the following structure:

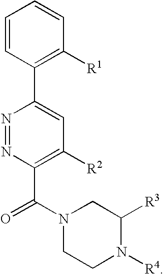

In the above structure, R¹ is independently selected from the group of radicals consisting of —(C₁-C₉ alkyl), —CH₂(C₃-C₈ cycloalkyl), —O(C₁-C₉ alkyl), and —OCH₂(C₃-C₈ cycloalkyl). R² is independently selected from the group of radicals consisting of —(C₁-C₉ alkyl), —CH₂(C₃-C₈ cycloalkyl), and —CH₂(C₆-C₁₀ aryl). R³ is selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof. R⁴ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH. Preferred species of this third aspect of the invention are represented by the following structures:

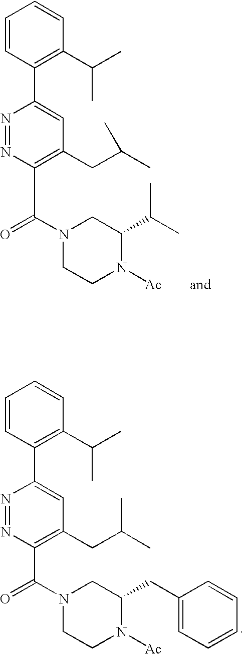

A fourth aspect of the invention is directed to another nonpeptidic mimetic of the i, i+3 or i+4, and i+7 positions of a peptide alpha-helix. This nonpeptidic mimetic is represented by Formula (IV):

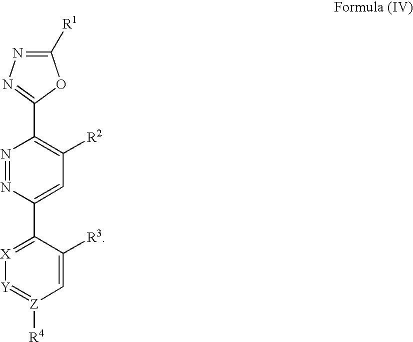

In Formula (IV), R¹ is a side chain of a naturally occurring amino acid or homologs thereof corresponding to the i position of the peptide alpha helix, or, alternatively is selected from the group of radicals consisting of —(C₁-C₉ alkyl), —CH₂(C₃-C₈ cycloalkyl), and —CH₂(C₆-C₁₀ aryl). R² is a side chain of a naturally occurring amino acid or homolog thereof corresponding to the i+3 or i+4 position of the peptide alpha helix, or, alternatively, is selected from the group of radicals consisting of —(C₁-C₉ alkyl), —CH₂(C₃-C₈ cycloalkyl), and —CH₂(C₆-C₁₀ aryl). R³ is a side chain of a naturally occurring amino acid or homolog thereof corresponding to the i+7 position of the peptide alpha helix, or, alternatively, is selected from the group of radicals consisting of —(C₁-C₉ alkyl), —(C₃-C₈ cycloalkyl), —O(C₁-C₉ alkyl), and —O(C₃-C₈ cycloalkyl). X, Y, and Z are independently selected from the group consisting of C and N. R⁴ is either absent or selected from the group of radicals consisting of —H, —(C₁-C₆ alkylene)COOH, —(C₃-C₈ cycloalkylene) COOH, —C(O)(C₁-C₆ alkylene)COOH, —C(O)(C₃-C₈ cycloalkylene)COOH, —NHC(O)(C₁-C₉ alkylene)COOH, and —NH(C₁-C₉ alkylene)COOH. However, there is a proviso that, at most, only one of the side chains of the naturally occurring amino acids or homolog thereof corresponding to the i, i+3 or i+4, and i+7 positions of the peptide alpha-helix can be hydrogen. In a preferred embodiment, the nonpeptidic mimetic is represented by the following structure:

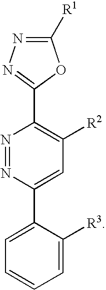

In the above structure, R¹ is selected from the group of radicals consisting of —(C₁-C₉ alkyl), —CH₂(C₃-C₈ cycloalkyl), and —CH₂(C₆-C₁₀ aryl). R² is selected from the group of radicals consisting of —(C₁-C₉ alkyl), —CH₂(C₃-C₈ cycloalkyl), and —CH₂(C₆-C₁₀ aryl). R³ is independently selected from the group of radicals consisting of —(C₁-C₉ alkyl), —(C₃-C₈ cycloalkyl), —O(C₁-C₉ alkyl), and —O(C₃-C₈ cycloalkyl). A preferred set species of this fourth aspect of the invention are represented by the following structure:

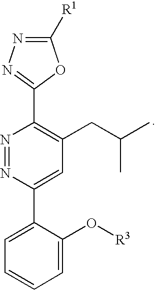

In the above structure, R¹ is selected from the group of radicals consisting of -i-Pr and —CH₂Ph. R³ is selected from the group of radicals consisting of -i-Pr and -Ph. Further species are represented by the following structures:

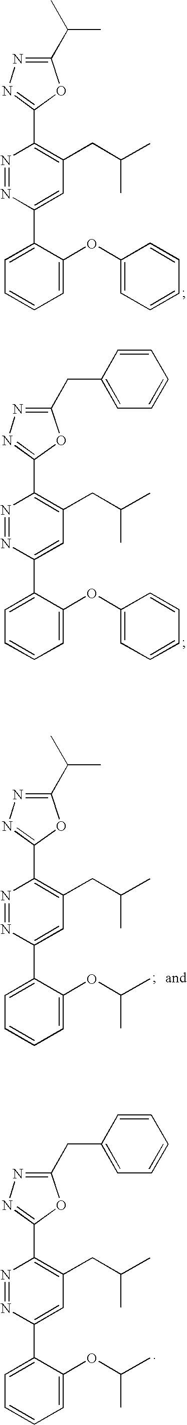

A fifth aspect of the invention is directed to another nonpeptidic mimetic of the i, i+3 or i+4, and i+7 positions of a peptide alpha-helix. The nonpeptidic mimetic is represented by Formula (V):

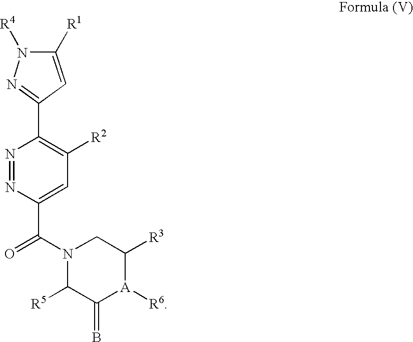

In Formula (V), at least one of R¹ and R⁴ is a side chain of a naturally occurring amino acid or homologs thereof corresponding to the i position of the peptide alpha helix. R² is a side chain of a naturally occurring amino acid or homolog thereof corresponding to the i+3 or i+4 position of the peptide alpha helix, or, alternatively, is selected from the group of radicals consisting of —(C₁-C₉ alkyl), —CH₂(C₃-C₈ cycloalkyl), and —CH₂(C₆-C₁₀ aryl). At least one of R³ and R⁵ is a side chain of a naturally occurring amino acid or homolog thereof corresponding to the i+7 position of the peptide alpha helix. If R¹ is not a side chain of a naturally occurring amino acid or homolog thereof corresponding to the i position, then R¹ is selected from the group of radicals consisting of —(C₁-C₉ alkyl), —CH₂(C₃-C₈ cycloalkyl), and —CH₂(C₆-C₁₀ aryl). If R⁴ is not a side chain of a naturally occurring amino acid or homolog thereof corresponding to the i position, then R⁴ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH. If R³ is not a side chain of a naturally occurring amino acid or homolog thereof corresponding to the i+7 position, then R³ is a radical is selected from the group consisting of —H, —O(C₁-C₆ alkyl), and —OC(O)—(C₁-C₆ alkyl). R⁶ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH. A is selected from the group of di- or triradicals consisting of [═N(CH₃)—]⁺, ═N—, —O—, —CH₂—, and ═CH—. B is either —O— or (—H)₂. However, the following provisos apply. At least one of R¹ and R⁴ is a side chain of a naturally occurring amino acid or homolog thereof corresponding to the position of the peptide alpha helix. At least one of R³ and R⁵ is a side chain of a naturally occurring amino acid or homolog thereof corresponding to the i+7 position of the peptide alpha helix, If R⁶ is absent, then A is selected from the group of diradicals consisting of —O— and —CH₂—. At most, only one of the side chains of the naturally occurring amino acids or homolog thereof corresponding to the i, i+3 or i+4, and i+7 positions of the peptide alpha-helix can be hydrogen. In a preferred embodiment of this fifth aspect of the invention, the nonpeptidic mimetic is represented by the following structure:

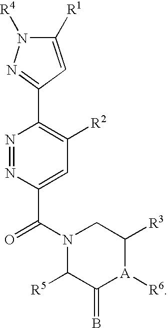

In the above structure, the side chain of the naturally occurring amino acid with respect to R¹, R², R³, R⁴, and R⁵ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof.

Another aspect of the invention is directed to methods for synthesizing the compounds of the first aspect and intermediates thereof.

Another aspect of the invention is directed to a process for disrupting a protein-protein interaction selected from the group consisting of Bak/Bcl-X_{L}, p53/HDM2, calmodulin/smooth muscle myosin light-chain kinase, and gp41 assembly comprising the step of contacting a compound of claim 1 with sufficient concentration to disrupt the protein-protein interaction.

Another aspect of the invention is directed to a process for treating conditions and/or disorders mediated by the disruption of the protein-protein interaction of claim 39 comprising the step of administering a sufficient amount to a compound of claim 1 to a patient to the disruption of the protein-protein interaction.

The synthesis of the desired α-helix mimetics has been performed in few steps. While specific derivatives are prepared and disclosed here, the methodology reported is applicable for a broader, more general decoration of the scaffold to provide a diversity of compounds within the scope of the invention. These compounds are disclosed to have utility, inter alia, as inhibitors of the protein-protein interactions discussed above.

### BRIEF DESCRIPTION OF DRAWINGS

FIG. 1 illustrates the derivation of four different amphilic, non-peptide scaffolds that mimic the presentation of i, i+3, or i+4, and i+7 residues of a peptide α-helix from a single intermediate.

FIG. 2 illustrates two different scaffolds and their superposition on top of the i, i+4, and i+7 positions of an α-helix.

FIG. 3 illustrates a retrosynthetic scheme showing how the compounds 1 and 2 can be derived from two different regioisomers 3 and 4 with a minimum number of carbon-carbon bond forming reactions.

FIG. 4 illustrates a scheme for the synthesis of pyrazole-pyridazine-piperazine scaffolds 1a-d.

FIG. 5 illustrates a scheme for the synthesis of pyrimidine-pyridazine-piperazine scaffold.

FIG. 6 illustrates a scheme showing the synthesis of the oxadiazole-pyridazine-phenyl scaffold.

FIG. 7 illustrates a scheme for the synthesis of piperazine-pyridazine-phenyl scaffold from intermediate 4.

### DETAILED DESCRIPTION

The synthesis of new α-helix scaffolds mimicking i, i+3 or i+4, i+7 residues, was accomplished. The common pyridazine heterocycle originates from the easily available building block, 6. These scaffolds may be thought of as synthetic counterparts of amphiphilic α-helices having a “wet face” along one side and a hydrophobic face along the other side of the helix.

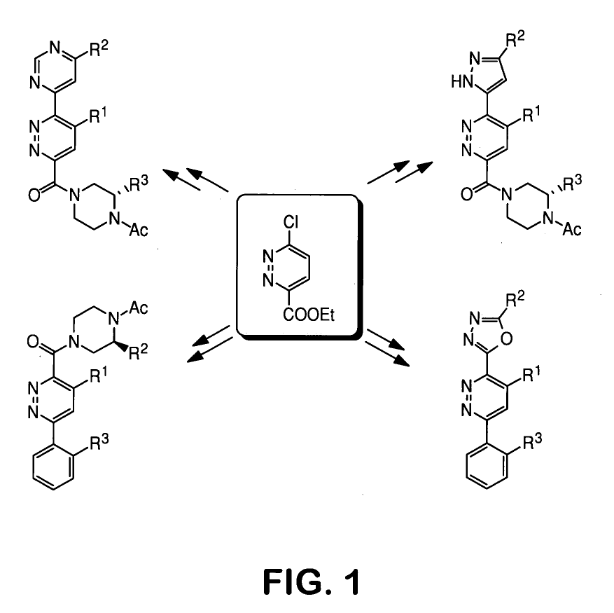

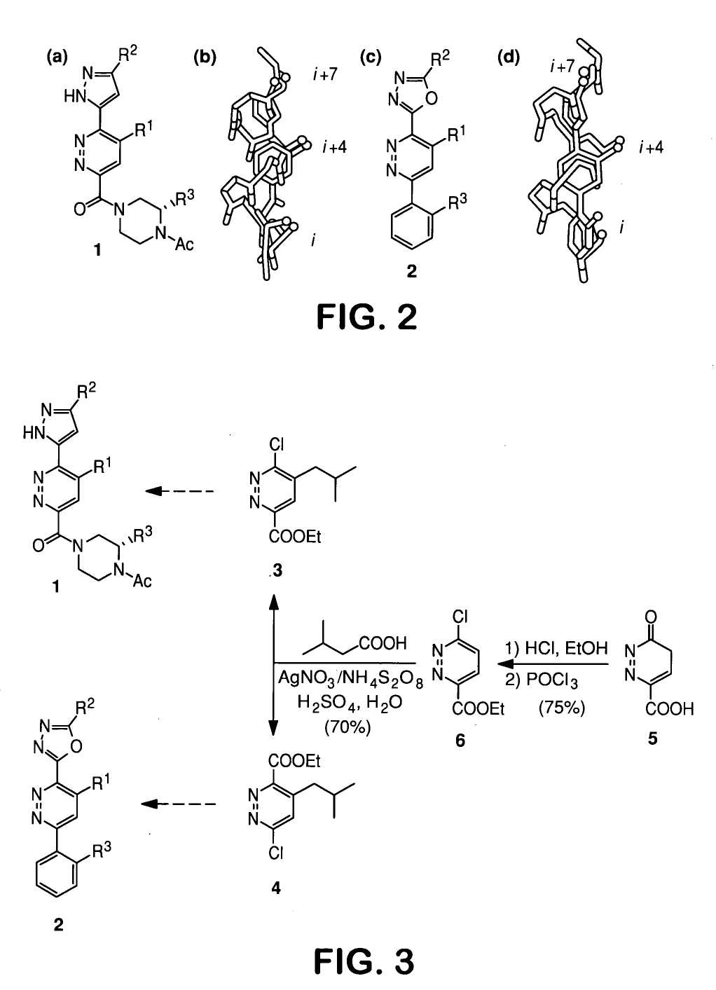

Here is described the synthesis of small libraries of new classes of low-molecular-weight α-helix mimetics having a pyridazine ring in the central position and hydrophobic amino-acid side chains of the key i, i+3 or i+4, i+7 positions. The derivation of all four structures from a common starting material is shown in FIG. 1. These include the pyrazole-pyridazine-piperazine scaffold 1 and the oxadiazole-pyridazine-phenyl scaffold 2 (FIG. 2).

What was sought was an improved synthetic accessibity, and an amphiphilic structure with hydrophobic surface for recognition and a “wet edge” for enhanced solubility.

As depicted in FIG. 3, compounds 1 and 2 were obtained in few steps involving a minimum number of C—C bond forming reactions, starting from two regioisomeric 4- and 5-alkyl-3-chloro-6-carboxypyridazine ethyl esters 3 and 4, respectively. The latter was be obtained by nucleophilic alkylation of 3-chloro-6-carboxypyridazine ethyl ester 6 by alkyl free radicals that are known to react with electron-poor protonated heteroaromatics such as, for instance, 3,6-dichloropyridazine (Samaritoni, J. G. Org. Prep. Proc. Int. 1988, 20, 117-121). Due to the electronic properties of the substituents, the electrophilicity of C-4 and C-5 on 6 should not differ so much. Accordingly, esterification of commercially available 6-oxo-1,6-dihydropyridazine-3-carboxylic acid 5 followed by treatment with POCl₃ gave 6 (Morishita, M., et al. Chem. Pharm. Bull. 1994, 42, 371-372). This underwent homolytic alkylation by free iso-butyl radical, generated by silver-catalyzed oxidative decarboxylation of iso-valeric acid, and led to a mixture (ca 2:1 regioisomeric ratio) of regioisomers 3 and 4 that were easily separated by flash chromatography.

The structures of regioisomers 3 and 4 were assigned on the basis of the chemical shifts of the aromatic protons (see Supporting Information). Moreover, it has been reported that a similar pyridazine having a carbonitrile group instead of the ethyl ester function reacted with pivalic acid under the same conditions to yield a 7:3 mixture of two regioisomers, the major one having the same regiochemistry (confirmed by X-ray analysis) of 3 (Hackler, R. E.; et al. J. Agric. Food Chem. 1990, 38, 508-514).

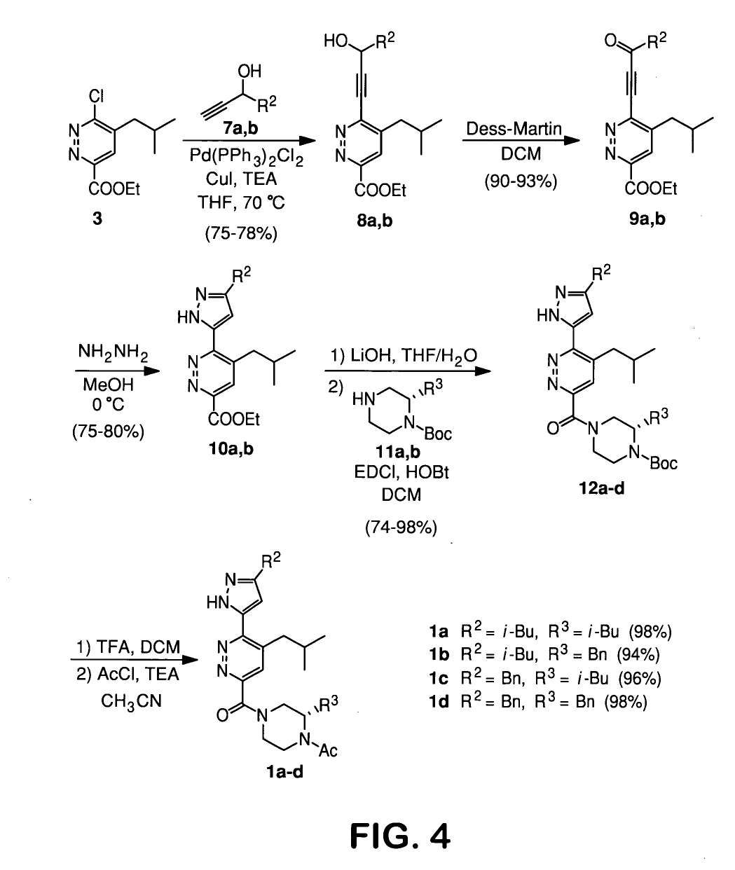

The major regioisomer 3, underwent Sonogashira coupling (For a very recent review on Sonogashira coupling see: Chinchilla, R.; Nájera, C. Chem. Rev. 2007, 107, 874-922 and references cited therein.) with benzyl and iso-butyl alkynyl alcohols 7a,b (Benzyl and iso-butyl alkynyl alcohols 7a,b were obtained in excellent yields by reacting the corresponding aldehydes with ethynylmagnesium bromide.) and eventually led to pyridazines 8a,b respectively, in good yields (FIG. 4). Oxidation of 8a,b to the corresponding ketones 9a,b was achieved in high yields with the Dess-Martin periodinane reagent, while oxidation with MnO₂ gave good results only with compound 8a (R²=iso-butyl). Acetylenic ketones such as 9 are known to undergo heteroannulation reactions with bis-nucleophiles like ureas, guanidines, hydrazines and others (Bagley, M. C.; et al. Synlett 2003, 259-262). Accordingly, compounds 9a,b were reacted with hydrazine in MeOH at 0° C. affording after 1 h the pyrazole derivatives 10a,b in high yields. After hydrolysis of the ethyl ester function with LiOH followed by coupling with different commercially available N-Boc-protected piperazines 11a,b, we obtained a small library of compounds 12a-d. After deprotection, these were selectively acetylated at the free amine function of the piperazine ring leading to the target compounds 1a-d.

Such structures were recently shown to give good overlap of their protruding functions with the side chains of α-helices (Biros, S. M.; et al. Bioorg. Med. Chem. Lett. 2007, 17, 4641-4645).

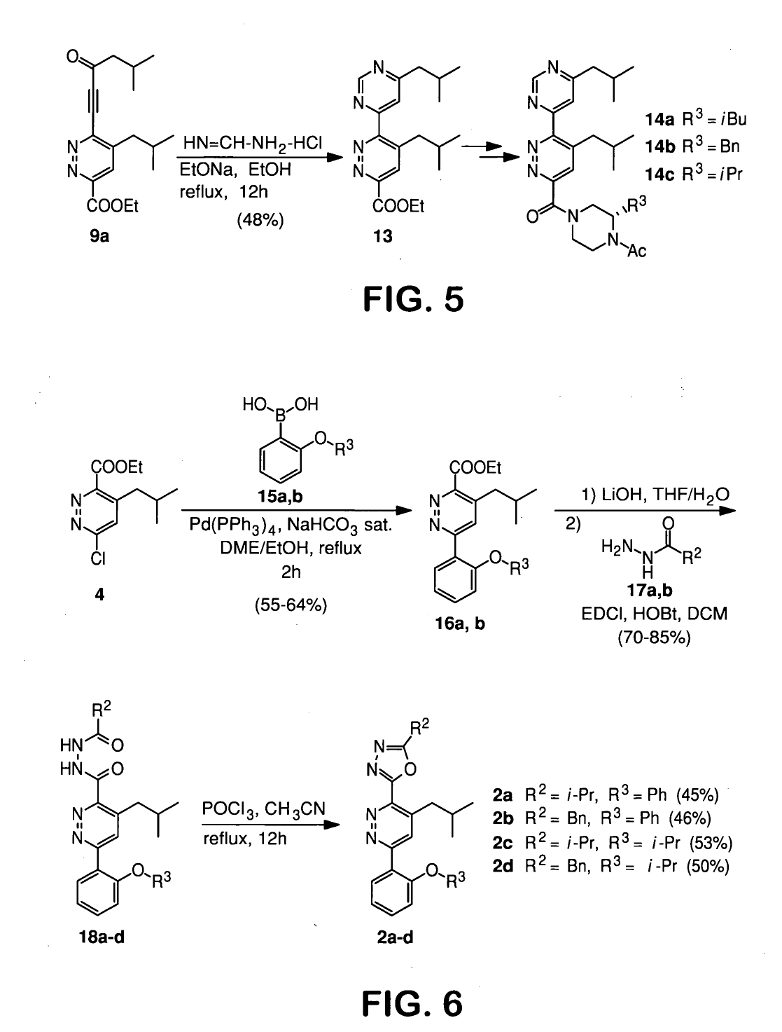

The versatility of alkynyl ketone 9a was further exploited in its reaction with formamidine in refluxing EtOH leading to the formation of the pyrimidine derivative 13 in moderate yield (FIG. 5). Following the same strategy depicted in FIG. 4, a new class of α-helix mimetics was synthesized, namely the pyrimidine-pyridazine-piperazine scaffold (compounds 14a-c).

The minor regioisomer 4 was found to be sufficiently electron poor to undergo Suzuki coupling (Miyaura, N.; Suzuki, A. Chem. Rev. 1995, 95, 2457-2483) with commercially available 2-alkoxyaryl boronic acids 15a,b affording compounds 16a,b in acceptable yields (FIG. 6). Both the alkoxy side chains and alkyl side chains serve to mimic the key hydrophobic residues in protein-a-helix ligand interactions (Ernst, J. T.; et al. Angew. Chem. Int. Ed. 2003, 42, 535-539). Hydrolysis of the ethyl ester with LiOH, followed by coupling with N-acyl hydrazides 17a,b (easily obtained by reaction between hydrazine and the corresponding esters) mediated by EDCI/HOBt led to the formation of intermediates 18a-d in good overall yields. Finally, N,N′-diacyl hydrazides 18a-d were dehydrated using POCl₃ in refluxing CH₃CN to achieve the synthesis of α-helix mimetic oxadiazole-pyridazine-phenyl scaffold 2.

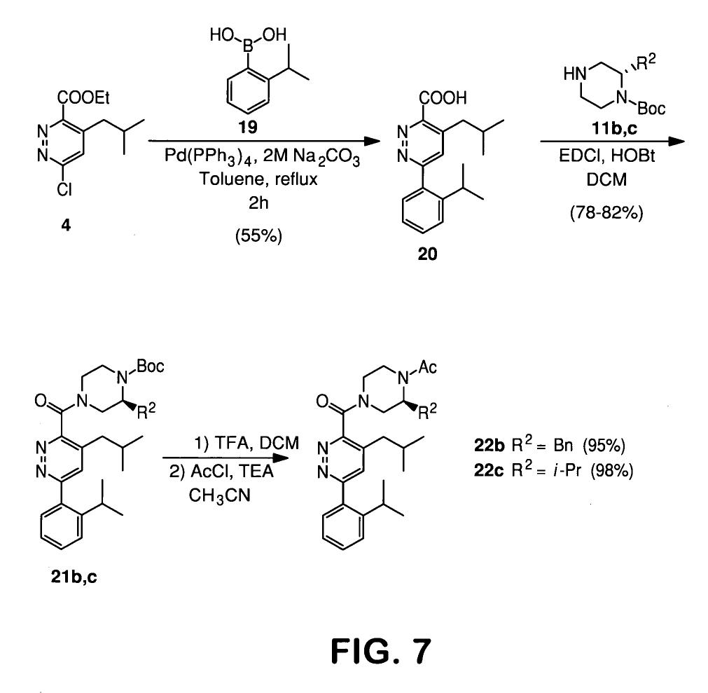

The Suzuki coupling with 2-iso-propylphenyl boronic acid 19 used a 2M aqueous solution of Na₂CO₃ (instead of a saturated aqueous solution of NaHCO₃) gave directly the free carboxylic acid 20. It could be used as intermediate for the construction of other scaffolds (FIG. 7). For example, coupling 20 with N-Boc-piperazines 11b,c gave, after the usual deprotection/acetylation sequence, the piperazine-pyridazine-phenyl scaffold, represented by compounds 22b,c.

### Synthetic Protocols:

General Methods Commercially available reagent-grade solvents were employed without purification. ¹H and ¹³C NMR spectra were recorded on 300 or 600 MHz spectrometers. Chemical shifts are expressed in ppm (δ), using tetramethylsilane (TMS) as internal standard for ¹H and ¹³C nuclei (δ_{H} and δ_{C}=0.00).

Materials: Key intermediate 6 was obtained according to the literature (Morishita, M., et al. Chem. Pharm. Bull. 1994, 42, 371-372.). Benzyl and iso-butyl alkynyl alcohols 7a,b were obtained by reacting the corresponding aldehydes with ethynylmagnesium bromide and their ¹H NMR and ¹³C NMR spectral data were in agreement with those previously reported (Kumar, M. P.; Liu, R.-S. J. Org. Chem. 2006, 71, 4951-4955; Fleming, S. A.; Liu, R.; Redd, J. T. Tetrahedron Lett 2005, 46, 8095-8098.). N-acyl hydrazides 17a,b were obtained by reaction between hydrazine and the corresponding esters and their ¹H NMR and ¹³C NMR spectral data were in agreement with those previously reported (Khan, K. M.; et al. Bioorg. Med. Chem. 2003, 11, 1381-1387.).

Homolytic radical alkylation of 3-chloro-6-carboxypyridazine ethyl ester 6. To a suspension of 3-chloro-6-carboxypyridazine ethyl ester 6 (1.86 g, 10 mmol) in distilled water (30 mL) iso-butyl carboxylic acid (2.1 mL, 2.25 mmol), conc. H₂SO₄ (0.8 mL, 15 mmol) and AgNO₃ (169 mg, 1 mmol) were added at room temperature. The mixture was heated at 65-75° C. and a solution of NH₄S₂O₈ (3.4 g, 15 mmol) in distilled water (10 mL) was added drop-wise in 10-15 minutes. The reaction was stirred for additional 30 minutes at 70-75° C., then poured in ice, neutralized with a 30% aqueous solution of NH₄OH and immediately extracted twice with dichloromethane. The collected organic layers were dried over magnesium sulfate, the solvent removed under reduced pressure and the crude material purified by flash chromatography to give 1.13 g of regioisomer 3 and 564 mg of regioisomer 4.

Ethyl-6-chloro-5-iso-butylpyridazine-3-carboxylate 3: R_{f}=0.49 (Hexane/AcOEt=70:30).

Ethyl-6-chloro-4-iso-butylpyridazine-3-carboxylate 4: R_{f}=0.55 (Hexane/AcOEt=70:30).

Sonogashira coupling on ethyl-6-chloro-5-iso-butyl pyridazine-3-carboxylate 3. General procedure. To a stirred solution of ethyl-6-chloro-5-iso-butylpyridazine-3-carboxylate 3 (1 equiv.) in dry THF (0.5 M solution) ethynyl alcohol (1.25 equiv.), dry TEA (2.7 equiv.), CuI (0.03 equiv.) and Pd(PPh₃)₂Cl₂ (0.03 equiv.) were added at room temperature under nitrogen atmosphere. The reaction was heated at 70° C. and stirred for 2 hours. The suspension was cooled to room temperature, diluted with AcOEt, filtered, the solvent removed under reduced pressure and the crude purified by flash chromatography.

Ethyl-6-(3-hydroxy-5-methylhex-1-ynyl)-5-iso-butylpyridazine-3-carboxylate 8a: ESI (m/z) 341 [M⁺+Na, (32)], 319 [M⁺+1, (100)], 121 (89).

Ethyl-6-(3-hydroxy-4-phenylbut-1-ynyl)-5-iso-butylpyridazine-3-carboxylate 8b: ESI (m/z) 375 [M⁺+Na, (32)], 353 [M⁺+1, (72)], 121 (100).

Oxidation of alkynyl alcohols 8a,b. General procedure. To a solution of alkynyl alcohol (1 equiv.) in dichloromethane (0.07 M solution) Dess-Martin periodinane (1.1 equiv.) was added at room temperature. The mixture was stirred overnight, filtered, the solvent removed under reduced pressure and the crude material purified by flash chromatography.

Ethyl-5-iso-butyl-6-(5-methyl-3-oxohex-1-ynyl)pyridazine-3-carboxylate 9a: R_{f}=0.17 (Hexane/AcOEt=80:20.

Ethyl-5-iso-butyl-6-(3-oxo-4-phenylbut-1-ynyl)pyridazine-3-carboxylate 9b: ESI (m/z) 373 [M⁺+Na, (6)], 351 [M⁺+1, (100)].

Synthesis of pyrazole derivatives 10a,b. General procedure. To a solution of propargyl ketone (1 equiv.) in MeOH (0.2 M solution) hydrazine hydrate (1 equiv.) was added at 0° C. The solution was stirred at 0° C. for one hour, heated to room temperature, the solvent removed under reduced pressure and the crude purified by flash chromatography.

Ethyl-5-iso-butyl-6-(3-iso-butyl-1H-pyrazol-5-yl)pyridazine-3-carboxylate 10a: ESI (m/z) 353 [M⁺+Na, (12)], 331 [M⁺+1, (100)].

Ethyl-6-(3-benzyl-1H-pyrazol-5-yl)-5-iso-butylpyridazine-3-carboxylate 10b: ESI (m/z) 387 [M⁺+Na, (16)], 365 [M⁺+1, (100)].

Synthesis of pyrimidine derivative 13. To a solution of sodium ethoxide (1.3 equiv.) in absolute ethanol (0.25 M solution), formamidine hydrochloride (1.3 equiv.) was added at room temperature under nitrogen atmosphere. The resulting suspension was stirred for 30 minutes. A solution of alkynyl ketone (1 equiv.) in a minimal amount of absolute ethanol was added and the suspension was refluxed overnight. The mixture was cooled to room temperature, filtered, the solvent removed under reduced pressure and the crude purified by flash chromatography. Ethyl-5-iso-butyl-6-(6-iso-butylpyrimidin-4-yl)pyridazine-3-carboxylate 13: R_{f}=0.17 (Hexane/AcOEt=80:20).

Hydrolysis of the ethyl esters 10a,b and 13. General procedure. To a solution of ethyl ester (1 equiv.) in a 4:1 mixture of THF/H₂O (0.05 M solution) LiOH hydrate (1.2 equiv.) was added at 0° C. After the hydrolysis is complete (TLC monitoring) the solution was acidified carefully with a 1N HCl aqueous solution, heated to room temperature and extracted with AcOEt. The collected organic layers were dried over magnesium sulfate, filtered and the solvent removed under reduced pressure leading to the free carboxylic acid that was used without any further purification.

Coupling leading to the scaffolds 12a-d and 21b,c. General procedure.

To a solution of the free carboxylic acid (1 equiv.) in dry dichloromethane (0.05 M solution) (S)—N-1-Boc-2-benzylpiperazine (1.05 equiv.), EDCI (1.1 equiv.), HOBt (1.1 equiv.) and DIPEA (2 equiv.) were added at room temperature. The reaction was stirred overnight, the solvent evaporated under reduced pressure and the crude material purified by flash chromatography.

(S)-tert-butyl-2-iso-butyl-4-(5-iso-butyl-6-(3-iso-butyl-1H-pyrazol-5-yl)pyridazine-3-carbonyl)piperazine-1-carboxylate 12a: R_{f}=0.46 (CH₂Cl₂/MeOH=95:5).

(S)-tert-butyl-2-benzyl-4-[5-iso-butyl-6-(3-iso-butyl-1H-pyrazol-5-yl)pyridazine-3-carbonyl]piperazine-1-carboxylate 12b: R_{f}=0.21 (CH₂Cl₂/MeOH=98:2).

(S)-tert-butyl-4-(6-(3-benzyl-1H-pyrazol-5-yl)-5-iso-butylpyridazine-3-carbonyl)-2-isobutyl piperazine-1-carboxylate 12c: R_{f}=0.38 (CH₂Cl₂/MeOH=95:5).

(S)-tert-butyl-2-benzyl-4-[6-(3-benzyl-1H-pyrazol-5-yl)-5-iso-butylpyridazine-3-carbonyl]piperazine-1-carboxylate 12d: R_{f}=0.16 (CH₂Cl₂/MeOH=98:2).

(S)-tert-butyl-2-benzyl-4-[4-iso-butyl-6-(2-iso-propylphenyl)pyridazine-3-carbonyl]piperazine-1-carboxylate 21b: R_{f}=0.33 (CH₂Cl₂/MeOH=98:2).

(S)-tert-butyl-4-(4-iso-butyl-6-(2-iso-propyl phenyl)pyridazine-3-carbonyl)-2-iso-propylpiperazine-1-carboxylate 21c: R_{f}=0.45 (CH₂Cl₂/MeOH=95:5).

Suzuki coupling leading esters 16a,b. General procedure. To a solution of 4 (1 equiv.) in dry DME (0.05 M solution), Pd(PPh₃)₄ (0.07 equiv.) was added and the mixture stirred for 15 minutes under N₂ atmosphere. A solution of 2-(isopropyloxyphenyl)-boronic acid (1.1 equiv.) in a minimal amount of EtOH was added followed by a saturated aqueous NaHCO₃ solution (⅓ of the DME volume). The mixture was refluxed under a nitrogen atmosphere for 2 h, cooled to room temperature, and extracted with DCM. The collected organic layers were dried over magnesium sulfate, filtered, the solvent removed under reduced pressure and the crude material purified by flash chromatography.

Ethyl 4-iso-butyl-6-(2-phenoxyphenyl)pyridazine-3-carboxylate 16a: R_{f}=0.42 (Hexane/AcOEt=80:20).

Ethyl 4-iso-butyl-6-(2-iso-propoxyphenyl)pyridazine-3-carboxylate 16b: R_{f}=0.35 (Hexane/AcOEt=70:30).

Suzuki coupling leading carboxylic acid 20. A solution of 4 (172 mg, 0.71 mmol), 2-iso-propyl-phenylboronic acid (175 mg, 1.5 mmol), Pd(PPh₃)₄ (25 mg, 0.03 mmol), 2M aqueous Na₂CO₃ (0.750 mL, 2.1 mmol) in dry toluene (4.5 mL) was flushed with nitrogen for 5 minutes. The mixture was refluxed under nitrogen atmosphere overnight, cooled to room temperature, diluted with water and extracted with AcOEt. The collected organic layers were dried over magnesium sulfate, filtered, the solvent removed under reduced pressure and the crude material purified by flash chromatography affording 116 mg of 20.

4-iso-butyl-6-(2-iso-propylphenyl)pyridazine-3-carboxylic acid 20: ESI (m/z) 321 [M⁺+Na, (18)], 299 [M⁺+1, (100)].

Synthesis of bis-acylhydrazides 18a-d. General procedure. To a solution of ethyl ester (1 equiv.) in a 4:1 mixture of THF/H₂O (0.05 M solution) LiOH hydrate (1.2 equiv.) was added at 0° C. After the hydrolysis is complete (TLC monitoring) the solution was acidified carefully with a 1N HCl aqueous solution, heated to room temperature and extracted with AcOEt. The collected organic layers were dried over magnesium sulfate, filtered and the solvent removed under reduced pressure leading to the free carboxylic acid that was used without any further purification. To a solution of the free carboxylic acid in dry dichloromethane (0.05 M solution) N-acylhydrazine (1.05 equiv.), EDCI (1.1 equiv.), HOBt (1.1 equiv.) and DIPEA (2 equiv.) were added at room temperature. The reaction was stirred overnight, the solvent evaporated under reduced pressure and the crude purified by flash chromatography.

4-Iso-butyl-N′-iso-butyryl-6-(2-phenoxyphenyl)pyridazine-3-carbohydrazide 18a: R_{f}=0.47 (CH₂Cl₂/MeOH=95:5).

4-Iso-butyl-6-(2-phenoxyphenyl)-N′-(2-phenylacetyl)pyridazine-3-carbohydrazide 18b: R_{f}=0.42 (CH₂Cl₂/MeOH=95:5).

4-Iso-butyl-NM-iso-butyryl-6-(2-iso-propoxyphenyl)pyridazine-3-carbohydrazide 18c: R_{f}=0.35 (CH₂Cl₂/MeOH=95:5).

4-Iso-butyl-6-(2-iso-propoxyphenyl)-N′-(2-phenylacetyl)pyridazine-3-carbohydrazide 18d: R_{f}=0.35 (CH₂Cl₂/MeOH=95:5).

Synthesis of scaffold 2a-d. General procedure: To a solution of bis-acylhydrazide (1 equiv.) in dry CH₃CN (0.1 M solution) POCl₃ (12 equiv.) was added drop-wise. The mixture was refluxed 12 h, cooled to room temperature, poured in ice, made basic with saturated aqueous NaHCO₃ and extracted with AcOEt. The collected organic layers were dried over magnesium sulfate, filtered, the solvent removed under reduced pressure and the crude purified by flash chromatography.

2-(4-Iso-butyl-6-(2-phenoxyphenyl)pyridazin-3-yl)-5-iso-propyl-1,3,4-oxadiazole 2a: R_{f}=0.38 (Hexane/AcOEt=70:30).

2-Benzyl-5-(4-iso-butyl-6-(2-phenoxyphenyl)pyridazin-3-yl)-1,3,4-oxadiazole 2b: R_{f}=0.65 (Hexane/AcOEt=60:40).

2-(4-iso-butyl-6-(2-iso-propoxyphenyl)pyridazin-3-yl)-5-iso-propyl-1,3,4-oxadiazole 2c: R_{f}=0.33 (Hexane/AcOEt=70:30).

2-Benzyl-5-(4-iso-butyl-6-(2-iso-propoxyphenyl)pyridazin-3-yl)-1,3,4-oxadiazole 2d: R_{f}=0.35 (Hexane/AcOEt=70:30).

Synthesis of acetylated scaffolds 1a-d, 14a-c, 22a,b. General procedure: A solution of N-Boc protected α-helix mimic compound (1 equiv.) in a 10% solution of TFA in dry DCM (0.05 M solution) was stirred for 1 h at room temperature. The solution was made basic with saturated aqueous NaHCO₃ and extracted with DCM. The collected organic layers were dried over magnesium sulfate, filtered and the solvent removed under reduced pressure. The resulting free amine was dissolved in dry CH₃CN (0.01 M solution). Dry TEA (1.5 equiv.) was added followed by AcCl (1 equiv.) at rt. After the reaction was complete (TLC monitoring) the solvent was removed under reduced pressure and the crude purified by flash chromatography.

(S)-1-(2-iso-butyl-4-(5-iso-butyl-6-(3-iso-butyl-1H-pyrazol-5-yl)pyridazine-3-carbonyl)piperazin-1-yl)ethanone 1a: R_{f}=0.18 (AcOEt).

(S)-1-(4-(6-(3-benzyl-1H-pyrazol-5-yl)-5-iso-butyl pyridazine-3-carbonyl)-2-iso-butylpiperazin-1-yl)ethanone 1b: R_{f}=0.12 (AcOEt).

(S)-1-(2-benzyl-4-(5-iso-butyl-6-(3-iso-butyl-1H-pyrazol-5-yl)pyridazine-3-carbonyl)piperazin-1-yl)ethanone 1c: R_{f}=0.15 (AcOEt).

(S)-1-(2-benzyl-4-(6-(3-benzyl-1H-pyrazol-5-yl)-5-iso-butylpyridazine-3-carbonyl)piperazin-1-yl)ethanone 1d: R_{f}=0.16 (AcOEt).

(S)-1-(2-iso-butyl-4-(5-iso-butyl-6-(6-iso-butylpyrimidin-4-yl)pyridazine-3-carbonyl)piperazin-1-yl)ethanone 14a: R_{f}=0.21 (AcOEt).

(S)-1-(2-benzyl-4-(5-iso-butyl-6-(6-iso-butyl pyrimidin-4-yl)pyridazine-3-carbonyl)piperazin-1-yl)ethanone 14b: R_{f}=0.14 (AcOEt).

(S)-1-(4-(5-iso-butyl-6-(6-iso-butyl pyrimidin-4-yl)pyridazine-3-carbonyl)-2-iso-propylpiperazin-1-yl)ethanone 14c: R_{f}=0.20 (AcOEt).

(S)-1-(2-benzyl-4-(4-iso-butyl-6-(2-iso-propylphenyl)pyridazine-3-carbonyl)piperazin-1-yl)ethanone 22b: R_{f}=0.27 (AcOEt).

(S)-1-(4-(4-iso-butyl-6-(2-iso-propylphenyl)pyridazine-3-carbonyl)-2-iso-propylpiperazin-1-yl)ethanone 22c: R_{f}=0.53 (AcOEt).

### Definition of Terms

Side chains of amino acids are the groups attached to the alpha carbon of alpha-amino acids. For example the side chains of glycine, alanine, and phenylalanine are hydrogen, methyl, and benzyl, respectively. The side chains may be of any naturally occurring or synthetic alpha amino acid. Naturally occurring alpha amino acids include those found in naturally occurring peptides, proteins, hormones, neurotransmitters, and other naturally occurring molecules. Synthetic alpha amino acids include any non-naturally occurring amino acid known to those of skill in the art. Representative amino acids include, but are not limited to, glycine, alanine, serine, threonine, arginine, lysine, ornithine, aspartic acid, glutamic acid, asparagine, glutamine, phenylalanine, tyrosine, tryptophan, leucine, valine, isoleucine, cysteine, methionine, histidine, 4-trifluoromethyl-phenylalanine, 3-(2-pyridyl)-alanine, 3-(2-furyl)-alanine, 2,4-diaminobutyric acid, and the like.

Pharmaceutically acceptable salts include a salt with an inorganic base, organic base, inorganic acid, organic acid, or basic or acidic amino acid. As salts of inorganic bases, the invention includes, for example, alkali metals such as sodium or potassium, alkali earth metals such as calcium and magnesium or aluminum, and ammonia. As salts of organic bases, the invention includes, for example, trimethylamine, triethylamine, pyridine, picoline, ethanolamine, diethanolamine, triethanolamine. As salts of inorganic acids, the instant invention includes, for example, hydrochloric acid, boric acid, nitric acid, sulfuric acid, and phosphoric acid. As salts of organic acids, the instant invention includes, for example, formic acid, acetic acid, trifluoroacetic acid, fumaric acid, oxalic acid, tartaric acid, lactic acid, maleic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid.

Certain compounds within the scope of Formula I are derivatives referred to as prodrugs. The expression “prodrug” denotes a derivative of a known direct acting drug, e.g. esters and amides, which derivative has enhanced delivery characteristics and therapeutic value as compared to the drug, and is transformed into the active drug by an enzymatic or chemical process; see Notari, R. E., “Theory and Practice of Prodrug Kinetics,” Methods in Enzymology 112:309-323 (1985); Bodor, N., “Novel Approaches in Prodrug Design,” Drugs of the Future 6:165-182 (1981); and Bundgaard, H., “Design of Prodrugs: Bioreversible-Derivatives for Various Functional Groups and Chemical Entities,” in Design of Prodrugs (H. Bundgaard, ed.), Elsevier, New York (1985), Goodman and Gilmans, The Pharmacological Basis of Therapeutics, 8th ed., McGraw-Hill, Int. Ed. 1992. The preceding references are hereby incorporated by reference in their entirety.

Tautomers refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, ketones are typically in equilibrium with their enol forms. Thus, ketones and their enols are referred to as tautomers of each other. As readily understood by one skilled in the art, a wide variety of functional groups and other structures may exhibit tautomerism, and all tautomers of compounds having Formula I are within the scope of the present invention.

Compounds of the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

“Treating” within the context of the instant invention, means an alleviation, in whole or in part, of symptoms associated with a disorder or disease, or halt of further progression or worsening of those symptoms, or prevention or prophylaxis of the disease or disorder. Similarly, as used herein, a “therapeutically effective amount” of a compound of the invention refers to an amount of the compound that alleviates, in whole or in part, symptoms associated with a disorder or disease, or halts of further progression or worsening of those symptoms, or prevents or provides prophylaxis for the disease or disorder. Treatment may also include administering the pharmaceutical formulations of the present invention in combination with other therapies. For example, the compounds of the invention can also be administered in conjunction with other therapeutic agents against bone disease or agents used for the treatment of metabolic disorders.

### Detailed Description of Figures

FIG. 1 shows the derivation of four different amphilic, non-peptide scaffolds that mimic the presentation of i, i+3 or i+4, and i+7 residues of a peptide α-helix from a single intermediate. The approach uses a pyridazine core, and the synthesis involves only a few steps and minimizes the number of C—C bond forming reactions. The versatility of the synthesis makes it suitable for the preparation of small libraries of low-molecular-weight α-helix mimetics that could be targeted to certain protein/protein interactions.

FIG. 2 shows two different scaffolds and their superposition on top of the i, i+4, and i+7 positions of an α-helix. Structure (a) is a pyrazole-pyridazine-piperazine scaffold. The structure (b) is a stereo view of the superposition of I (orange in the original) on the i, i+4, i+7 positions of an α-helix. Structure (c) is an oxadiazole-pyridazine-phenyl scaffold. Structure (d) is a stereo view of the superposition of 2 (orange in the original) on the i, i+4, i+7 positions of an α-helix.

FIG. 3 is a scheme showing the synthesis of regioisomeric pyrimidines 3 and 4. Esterification of commercially available 6-oxo-1,6-dihydropyridazine-3-carboxylic acid 5 is followed by treatment with POCl₃ to give the electron-poor 3-chloro-6-carboxypyridazine ethyl ester 6. Nucleophilic alkylation of the heteroaromatic ring was accomplished by generating the free iso-butyl radical by silver catalyzed oxidative decarboxylation of iso-valeric acid. A mixture of regioisomers were obtained in a 2:1 ratio with the major isomer being 3. This was confirmed by X-ray crystallography. The isomers were readily separated by flash chromatography.

FIG. 4 is a scheme for the synthesis of pyrazole-pyridazine-piperazine scaffolds 1a-d. Starting with the major regioisomer 3, Sonogashira coupling (Review: Chinchilla, R.; Nájera, C. Chem. Rev. 2007, 107, 874-922 and references cited therein.) with the accessible benzyl and iso-butyl alkynyl alcohols 7a,b. Oxidation of the resulting alcohols 8a,b with Dess-Martin periodinane led to the corresponding ketones 9a,b. Oxidants such as MnO₂ gave only useful results where R²=isobutyl. The bis-nucleophile, hydrazine, reacted readily with the α,β-acetylenic ketone to give the pyrazole compounds 10a,b. The other part of the scaffold was assembled by hydrolyzing the ethyl ester and then coupling the carboxylic acid to different commercially available protected piperazines 11a,b. The final steps in the synthesis of this library was the removal of the Boc protecting group by TFA in dichloromethane (DCM) and acetylating the revealed amino group to provide compounds 1a-d.

FIG. 5 is a scheme for the synthesis of pyrimidine-pyridazine-piperazine scaffold. Starting with the acetylenic ketone intermediate from FIG. 4, 9a is reacted with formamidine hydrochloride in refluxing ethanol and sodium ethoxide. The pyrimidine-pyridazine scaffold 13, is functionalized in the same manner as in FIG. 4 to give the library of compounds 14a, 14b, and 14c.

FIG. 6 is a scheme showing the synthesis of the oxadiazole-pyridazine-phenyl scaffold. The minor regioisomer 4, from FIG. 3, was sufficiently electron-poor to undergo Suzuki coupling with commercially available 2-alkoxyboronic acids 15a,b giving compounds 16a,b in acceptable yields. Hydrolysis of the ethyl esters followed by coupling with N-acyl hydrazides 17a,b mediated by EDCI, HOBt led to the formation of intermediates 18a-d in good overall yields. The N,N′-diacylhydrazides 18a-d were then dehydrated using POCl₃ in refluxing CH₃CN to give compounds 2a-d.

FIG. 7 is a scheme for the synthesis of piperazine-pyridazine-phenyl scaffold from intermediate 4. Again, the electron-poor intermediate 4 was used for a Suzuki coupling with 2-isopropylphenyl boronic acid 19 in 2M Na₂CO₃ which gave directly the free carboxylic acid 20. This is then used to acylate commercially available N-Boc-protected piperazines 11b,c mediated by EDCI/HOBt giving compounds 21b,c. Boc-deprotection with TFA in dichloromethane is followed by acetylation with acetyl chloride giving 22b and 22c in excellent yields.

What is claimed is:

1. A nonpeptidic mimetic of the i, i+3 or i+4, and i+7 positions of a peptide alpha-helix, the nonpeptidic mimetic being represented by Formula (I): 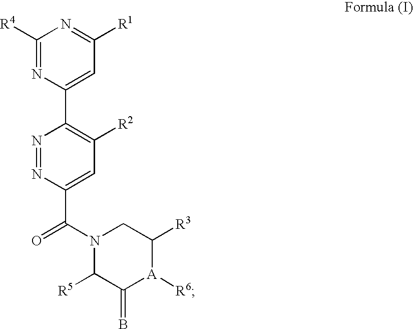
wherein:
at least one of R¹ and R⁴ is a side chain of a naturally occurring amino acid or homolog thereof corresponding to the i position of the peptide alpha helix;
R² is a side chain of a naturally occurring amino acid or homolog thereof corresponding to the i+3 or i+4 positions of the peptide alpha helix, or, alternatively is a radical selected from the group of radicals consisting of —(C₁-C₉ alkyl), —CH₂(C₃-C₈ cycloalkyl), and —CH₂(C₆-C₁₀ aryl);
at least one of R³ and R⁵ is a side chain of a naturally occurring amino acid or homolog thereof corresponding to the i+7 position of the peptide alpha helix;
if R¹ is not a side chain of a naturally occurring amino acid or homolog thereof corresponding to the i position of the peptide alpha helix, then R¹ is a radical selected from the group of radicals consisting of —H, —OH, —(C₁-C₈ alkyl), —CH₂(C₃-C₈ cycloalkyl), and —CH₂(C₆-C₁₀ aryl);
if R⁴ is not a side chain of a naturally occurring amino acid or homolog thereof corresponding to the i position of the peptide alpha helix, then R⁴ is selected from the group of radicals consisting of —H, —OH, —O(C₁-C₆ alkyl), —S—(C₁-C₆ alkyl), —NH—(C₁-C₆ alkyl), and —(C₁-C₆ alkyl);
if R³ is not a side chain of a naturally occurring amino acid or homolog thereof corresponding to the i+7 position, then R³ is a radical selected from the group consisting of hydrogen, —O(C₁-C₆ alkyl), and —OC(O)—(C₁-C₆ alkyl);
R⁶ is either absent or is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH;
A is selected from the group of di- or triradicals consisting of [═N(CH₃)—]⁺, ═N—, —O—, —CH₂—, and ═CH—; and
B is either —O— or (—H)₂;
with the following provisos:
at least one of R¹ and R⁴ is a side chain of a naturally occurring amino acid or homolog thereof corresponding to the i position of the peptide alpha helix;
at least one of R³ and R⁵ is a side chain of a naturally occurring amino acid or homolog thereof corresponding to the i+7 position of the peptide alpha helix;
if R⁶ is absent, then A is selected from the group of diradicals consisting of —O—, and —CH₂—; and
at most, only one of the side chains of the naturally occurring amino acids or homologs thereof corresponding to the i, i+3 or i+4, and i+7 positions of the peptide alpha-helix can be hydrogen.

2. A nonpeptidic mimetic according to claim 1 wherein:
the side chain of the naturally occurring amino acid with respect to R¹, R², R³, R⁴, and R⁵ is a radical independently selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof.

3. A nonpeptidic mimetic according to claim 2 represented by the following structure: 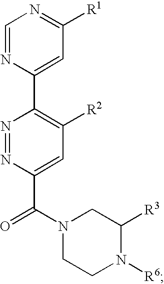
wherein:
R¹ and R² are radicals independently selected from the group of radicals consisting of —(C₁-C₉ alkyl), —CH₂(C₃-C₈ cycloalkyl), and —CH₂(C₆-C₁₀ aryl);
R³ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl); and
R⁶ is selected from the group of radicals consisting of —H, —(C₁-C₈ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH.

4. A nonpeptidic mimetic according to claim 3 represented by the following structure:

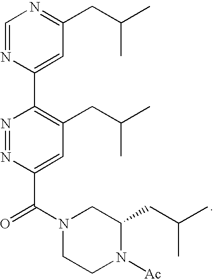

5. A nonpeptidic mimetic according to claim 3 represented by the following structure:

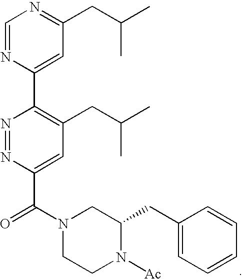

6. A nonpeptidic mimetic according to claim 3 represented by the following structure:

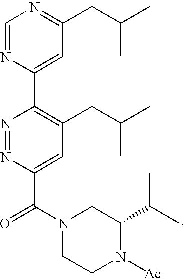

7. A nonpeptidic mimetic of the i, i+3 or i+4, and i+7 positions of a peptide alpha-helix, the nonpeptidic mimetic being represented by Formula (II): 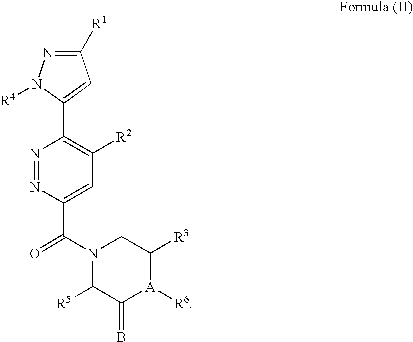
wherein:
at least one of R¹ and R⁴ is a side chain of a naturally occurring amino acid or homolog thereof corresponding to the i position of the peptide alpha helix;
R² is a side chain of a naturally occurring amino acid or homolog thereof corresponding to the i+3 or i+4 position of the peptide alpha helix, or alternatively, is a radical selected from the group consisting of —(C₁-C₉ alkyl), —CH₂(C₃-C₈ cycloalkyl), and —CH₂(C₆-C₁₀ aryl);
at least one of R³ and R⁵ is a side chain of a naturally occurring amino acid or homolog thereof corresponding to the i+7 position of the peptide alpha helix;
if R¹ is not a side chain of a naturally occurring amino acid or homolog thereof corresponding to the i position of the peptide alpha helix, then R¹ is a radical selected from the group of radicals consisting of —H, —OH, —(C₁-C₈ alkyl), —CH₂(C₃-C₈ cycloalkyl), and —CH₂(C₆-C₁₀ aryl);
if R⁴ is not a side chain of a naturally occurring amino acid or homolog thereof corresponding to the i position of the peptide alpha helix, then R⁴ is selected from the group of radicals consisting of —H, —(C₁-C₆ alkyl), —(C₁-C₆ alkylene)COOH, —C(O)(C₁-C₆ alkyl), —C(O)(C₁-C₆ alkylene)COOH;
if R³ is not a side chain of a naturally occurring amino acid or homolog thereof corresponding to the i+7 position of the peptide alpha helix, then R³ is a radical selected from the group consisting of —O(C₁-C₆ alkyl) and —OC(O)—(C₁-C₆ alkyl);
R⁶ is either absent or is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH;
A is selected from the group of di- or triradicals consisting of [═N(CH₃)—]⁺, ═N—, —O—, —CH₂—, and ═CH—; and
B is either —O— or (—H)₂;
with the following provisos:
at least one of R¹ and R⁴ is a side chain of a naturally occurring amino acid or homolog thereof corresponding to the i position of the peptide alpha helix;
at least one of R³ and R⁵ is a side chain of a naturally occurring amino acid or homolog thereof corresponding to the i+7 position of the peptide alpha helix;
if R⁶ is absent, then A is selected from the group of diradicals consisting of —O—, and —CH₂—; and
at most, only one of the side chains of the naturally occurring amino acids or homolog thereof corresponding to the i, i+3 or i+4, and i+7 positions of the peptide alpha-helix can be hydrogen.

8. A nonpeptidic mimetic according to claim 7 wherein:
the side chain of the naturally occurring amino acid with respect to R¹, R², R³, R⁴, and R⁵ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof.

9. A nonpeptidic mimetic according to claim 8 represented by the following structure: 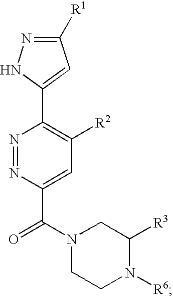
wherein:
R¹ and R² are radicals independently selected from the group of radicals consisting of —(C₁-C₉ alkyl), —CH₂(C₃-C₈ cycloalkyl), and —CH₂(C₆-C₁₀ aryl); and
R³ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof.

10. A nonpeptidic mimetic according to claim 9 represented by the following structure:

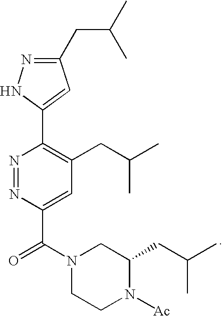

11. A nonpeptidic mimetic according to claim 9 represented by the following structure:

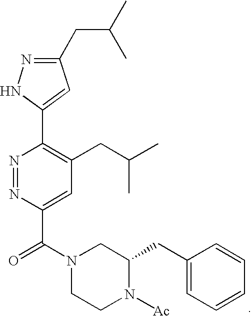

12. A nonpeptidic mimetic according to claim 9 represented by the following structure:

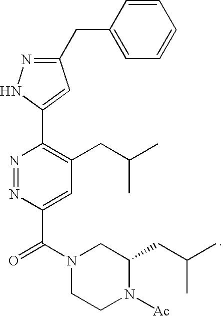

13. A nonpeptidic mimetic according to claim 9 represented by the following structure:

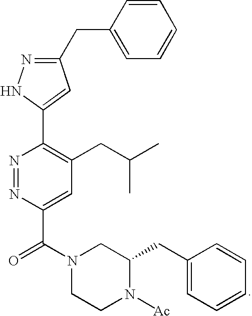

14. A nonpeptidic mimetic of the i, i+3 or i+4, and i+7 positions of a peptide alpha-helix, the nonpeptidic mimetic being represented by Formula (III): 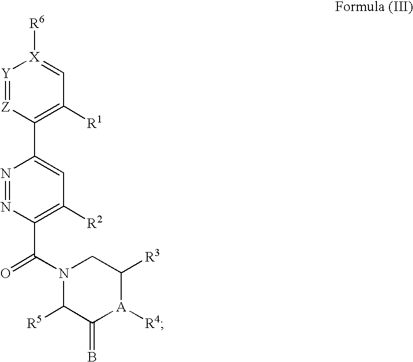
wherein:
at least one of R³ and R⁵ is a side chain of a naturally occurring amino acid or homolog thereof corresponding to the i position of the peptide alpha helix;
R² is a side chain of a naturally occurring amino acid or homolog thereof corresponding to the i+3 or i+4 position of the peptide alpha helix, or, alternatively, R² is selected from the group of radicals consisting of —(C₁-C₉ alkyl), —CH₂(C₃-C₈ cycloalkyl), and —CH₂(C₆-C₁₀ aryl);
R¹ is a side chain of a naturally occurring amino acid or homolog thereof corresponding to the i+7 position of the peptide alpha helix, or, alternatively, is selected from the group of radicals consisting of —(C₁-C₈ alkyl), —CH₂(C₃-C₈ cycloalkyl), —O(C₁-C₉ alkyl), and —OCH₂(C₃-C₈ cycloalkyl);
if R³ is not a side chain of a naturally occurring amino acid or homolog thereof corresponding to the i position of the peptide alpha helix, then R³ is a radical is selected from the group consisting of —O(C₁-C₆ alkyl) and —OC(O)—(C₁-C₆ alkyl);
R⁴ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH;
R⁶ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH;
X, Y, and Z are each independently selected from the group consisting of C and N;
A is selected from the group of di- or triradicals consisting of [═N(CH₃)—]⁺, ═N—, —O—, —CH₂—, and ═CH—; and
B is either —O— or (—H)₂;
with the following provisos:
at least one of R³ and R⁵ is a side chain of a naturally occurring amino acid or homolog thereof corresponding to the i position of the peptide alpha helix; and
if R⁶ is absent, then A is selected from the group of diradicals consisting of —O—, and —CH₂—; and
at most, only one of the side chains of the naturally occurring amino acids or homologs thereof corresponding to the i, i+3 or i+4, and i+7 positions of the peptide alpha-helix can be hydrogen.

15. A nonpeptidic mimetic according to claim 14 wherein:
the side chain of the naturally occurring amino acid from which R¹, R², R³, and R⁵ may be selected is a radical independently selected from the group consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl).

16. A nonpeptidic mimetic according to claim 15 represented by the following structure: 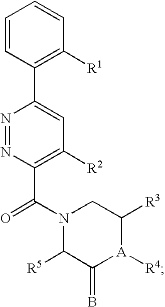
wherein:
R¹ is independently selected from the group of radicals consisting of —(C₁-C₈ alkyl), —CH₂(C₃-C₈ cycloalkyl), —O(C₁-C₉ alkyl), and —OCH₂(C₃-C₈ cycloalkyl);
R² is independently selected from the group of radicals consisting of —(C₁-C₉ alkyl), —CH₂(C₃-C₈ cycloalkyl), and —CH₂(C₆-C₁₀ aryl);
R³ is selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof;
R⁴ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH; and
R⁵ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, and homologs thereof.

17. A nonpeptidic mimetic according to claim 16 represented by the following structure: 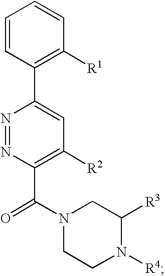
wherein:
R¹ is independently selected from the group of radicals consisting of —(C₁-C₉ alkyl), —CH₂(C₃-C₈ cycloalkyl), —O(C₁-C₉ alkyl), and —OCH₂(C₃-C₈ cycloalkyl);
R² is independently selected from the group of radicals consisting of —(C₁-C₉ alkyl), —CH₂(C₃-C₈ cycloalkyl), and —CH₂(C₆-C₁₀ aryl);
R³ is selected from the group of radicals consisting of —H, —CH₃, —CH₂CH₃, —CH(CH₃)₂, —CH₂CH(CH₃)₂1-CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof; and
R⁴ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH.

18. A nonpeptidic mimetic according to claim 17 represented by the following structure:

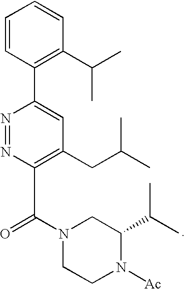

19. A nonpeptidic mimetic according to claim 18 represented by the following structure:

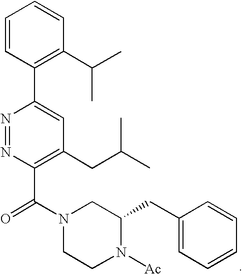

20. A nonpeptidic mimetic of the i, i+3 or i+4, and i+7 positions of a peptide alpha-helix, the nonpeptidic mimetic being represented by Formula (IV): 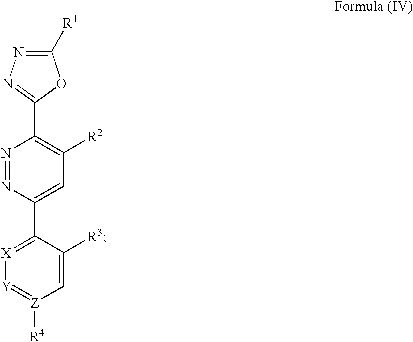
wherein:
R¹ is a side chain of a naturally occurring amino acid or homologs thereof corresponding to the i position of the peptide alpha helix, or, alternatively is selected from the group of radicals consisting of —(C₁-C₈ alkyl), —CH₂(C₃-C₈ cycloalkyl), and —CH₂(C₆-C₁₀ aryl);
R² is a side chain of a naturally occurring amino acid or homolog thereof corresponding to the i+3 or i+4 position of the peptide alpha helix, or, alternatively, is selected from the group of radicals consisting of —(C₁-C₉ alkyl), —CH₂(C₃-C₈ cycloalkyl), and —CH₂(C₆-C₁₀ aryl);
R³ is a side chain of a naturally occurring amino acid or homolog thereof corresponding to the i+7 position of the peptide alpha helix, or, alternatively, is selected from the group of radicals consisting of —(C₁-C₉ alkyl), —(C₃-C₈ cycloalkyl), —O(C₁-C₈ alkyl), and —O(C₃-C₈ cycloalkyl);
X, Y, and Z are independently selected from the group consisting of C and N; and
R⁴ is either absent or selected from the group of radicals consisting of —H, —(C₁-C₆ alkylene)COOH, —(C₃-C₈ cycloalkylene)COOH, —C(O)(C₁-C₆ alkylene)COOH, —C(O)(C₃-C₈ cycloalkylene)COOH, —NHC(O)(C₁-C₉ alkylene)COOH, and —NH(C₁-C₉ alkylene)COOH;
with a proviso that, at most, only one of the side chains of the naturally occurring amino acids or homolog thereof corresponding to the i, i+3 or i+4, and i+7 positions of the peptide alpha-helix can be hydrogen.

21. A nonpeptidic mimetic according to claim 20 represented by the following structure: 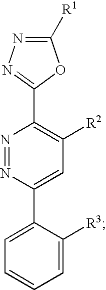
wherein:
R¹ is selected from the group of radicals consisting of —(C₁-C₉ alkyl), —CH₂(C₃-C₈ cycloalkyl), and —CH₂(C₆-C₁₀ aryl);
R² is selected from the group of radicals consisting of —(C₁-C₉ alkyl), —CH₂(C₃-C₈ cycloalkyl), and —CH₂(C₆-C₁₀ aryl); and
R³ is independently selected from the group of radicals consisting of —(C₁-C₉ alkyl), —(C₃-C₈ cycloalkyl), —O(C₁-C₉ alkyl), and —O(C₃-C₈ cycloalkyl).

22. A nonpeptidic mimetic according to claim 21 represented by the following structure: 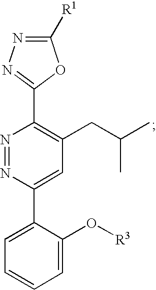
wherein
R¹ is selected from the group of radicals consisting of -i-Pr and —CH₂Ph; and
R³ is selected from the group of radicals consisting of -i-Pr and -Ph.

23. A nonpeptidic mimetic according to claim 22 represented by the following structure:

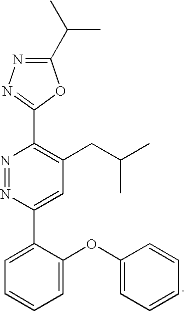

24. A nonpeptidic mimetic according to claim 22 represented by the following structure:

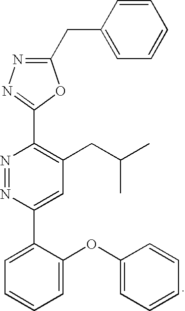

25. A nonpeptidic mimetic according to claim 22 represented by the following structure:

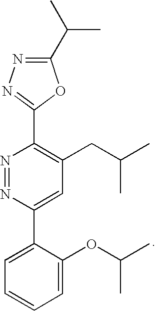

26. A nonpeptidic mimetic according to claim 22 represented by the following structure:

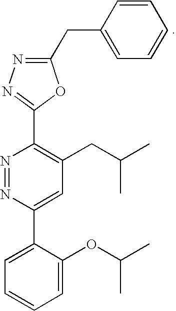

27. A nonpeptidic mimetic of the i, i+3 or i+4, and i+7 positions of a peptide alpha-helix, the nonpeptidic mimetic being represented by Formula (V): 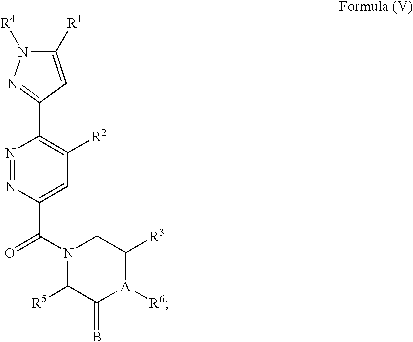
wherein:
at least one of R¹ and R⁴ is a side chain of a naturally occurring amino acid or homologs thereof corresponding to the i position of the peptide alpha helix;
R² is a side chain of a naturally occurring amino acid or homolog thereof corresponding to the i+3 or i+4 position of the peptide alpha helix, or, alternatively, is selected from the group of radicals consisting of —(C₁-C₉ alkyl), —CH₂(C₃-C₈ cycloalkyl), and —CH₂(C₆-C₁₀ aryl);
at least one of R³ and R⁵ is a side chain of a naturally occurring amino acid or homolog thereof corresponding to the i+7 position of the peptide alpha helix;
if R¹ is not a side chain of a naturally occurring amino acid or homolog thereof corresponding to the i position, then R¹ is selected from the group of radicals consisting of —(C₁-C₉ alkyl), —CH₂(C₃-C₈ cycloalkyl), and —CH₂(C₆-C₁₀ aryl);
if R⁴ is not a side chain of a naturally occurring amino acid or homolog thereof corresponding to the i position, then R⁴ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH;
if R³ is not a side chain of a naturally occurring amino acid or homolog thereof corresponding to the i+7 position, then R³ is a radical is selected from the group consisting of —H, —O(C₁-C₆ alkyl), and —OC(O)—(C₁-C₆ alkyl);
R⁶ is selected from the group of radicals consisting of —H, —(C₁-C₆ alkyl), —(C₃-C₈ cycloalkyl), —(C₁-C₆ alkylene)COOH, —(C₃-C₈ cycloalkylene)COOH, —C(O)(C₁-C₆ alkyl), —C(O)(C₃-C₈ cycloalkyl), —C(O)(C₁-C₆ alkylene)COOH, and —C(O)(C₃-C₈ cycloalkylene)COOH;
A is selected from the group of di- or triradicals consisting of [═N(CH₃)—]⁺, ═N—, —O—, —CH₂—, and ═CH—; and
B is either —O— or (—H)₂;
with the following provisos:
at least one of R¹ and R⁴ is a side chain of a naturally occurring amino acid or homolog thereof corresponding to the i position of the peptide alpha helix;
at least one of R³ and R⁵ is a side chain of a naturally occurring amino acid or homolog thereof corresponding to the i+7 position of the peptide alpha helix;
if R⁶ is absent, then A is selected from the group of diradicals consisting of —O— and —CH₂—; and
at most, only one of the side chains of the naturally occurring amino acids or homolog thereof corresponding to the i, i+3 or i+4, and i+7 positions of the peptide alpha-helix can be hydrogen.

28. A nonpeptidic mimetic according to claim 27 represented by the following structure: 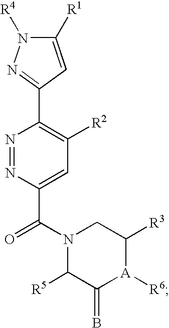
wherein:
the side chain of the naturally occurring amino acid with respect to R¹, R², R³, R⁴, and R⁵ is a radical selected from the group of radicals consisting of —H, —CH₃, —CH₂CH₃, —CH(CH₃)₂, —CH₂CH(CH₃)₂, —CH₂CH₂CH₂CH₃, —CH(CH₃)(CH₂CH₃), —CH₂OH, —CH₂SH, —CH₂CH₂SCH₃, —CH(OH)CH₃, —CH₂Ph, —CH₂C₆H₄OH, —CH₂C₆H₂I₂OH, —CH₂(3-indole), —CH₂CONH₂, —CH₂COOH, —CH₂CH₂CONH₂, —CH₂CH₂COOH, —CH₂CH₂CH₂CH₂NH₂, —CH₂(4-imidazole), —CH₂CH₂CH₂NHC(NH)NH₂, —O(C₁-C₆ alkyl), and OC(O)—(C₁-C₆ alkyl) and homologs thereof.

29. A process for synthesizing any of the compounds of claims 1-28 and intermediates thereof.

30. A process for disrupting a protein-protein interaction selected from the group consisting of Bak/Bcl-X_{L}, p53/HDM2, calmodulin/smooth muscle myosin light-chain kinase, and gp41 assembly comprising the step of contacting a compound of any of Formulas (I) — (V) with sufficient concentration to disrupt the protein-protein interaction.

31. A process for treating conditions and/or disorders mediated by the disruption of the protein-protein interaction of claim 30 comprising the step of administering a sufficient amount to a compound of any of Formulas (I)-(V) to a patient to the disruption of the protein-protein interaction.
